(19) United States
(12) Patent Application Publication
(10) Pub. No.: US 2021/0317302 A1
(43) Pub. Date: Oct. 14, 2021

(54) Biodegradable Polymer Composition and Method of Producing the Same

(71) Applicant: MOHARRAM VENTURES INC., London (CA)

(72) Inventors: Tarek Moharram, London, Ontario (CA); FAISAL SAHUL HAMEED, Leamington, Ontario (CA); Arshdeep Singh, London, Ontario (CA); Pathik Daxeshkumar Patel, Richmond Hill, Ontario (CA); Devon Brianne Gray, Kingston, Ontario (CA); Nicole Lindsay Kocher, London, Ontario (CA); Matthew Douglas Charles Hartin, London, Ontario (CA); Najwa Zebian, London, Ontario (CA)

(21) Appl. No.: 17/270,690

(22) PCT Filed: Aug. 23, 2019
(86) PCT No.: PCT/CA2019/000120

§ 371 Date: Feb. 23, 2021

Related U.S. Application Data
(60) Provisional application No. 62/722,535, filed on Aug. 24, 2018.

(51) Int. Cl. C08L 67/04; C08L 3/02; C08K 5/11; C08K 5/09; C08J 11/00; C12P 7/62

(52) U.S. Cl. CPC C08L 67/04; C08L 3/02; C08K 5/11; C08J 2367/04; C08J 11/00; C12P 7/625; C08K 5/09

(57) ABSTRACT

A biodegradable polymer composition, according to the present invention, comprises polyhydroxybutyrate and poly(3-hydroxybutyrate-co-3-hydroxyhexanoate) blended with thermoplastic starch, one or more compatibilizers selected from the group consisting of dihexyl sodium sulfosuccinate and maleic anhydride, and one or more additives selected from the group consisting of micro-crystalline cellulose and cellulose. Methods of producing a biodegradable polymer use processed cannabis waste as a carbon source.

### FIELD OF THE INVENTION

The present invention relates to biodegradable polymers, in particular, to biodegradable polymer compositions and methods of producing the same using cannabis waste as a carbon source.

### BACKGROUND

Plastic is a light-weight, durable, and versatile material and is an integral part of many industries from construction to healthcare, and from consumer goods to packaging materials. The production of many plastic materials relies on non-renewable resources, making the long-term viability both economically and environmentally unsustainable. The time required for environmental breakdown of many types of plastic has also aggravated these issues. Typically, plastics used in consumer items such as plastic straws take about 200 years to break down in the environment. More durable plastics, such as those used in fishing line can take as much as 600 years to break down.

As a result, environmental buildup of plastic waste has become an increasingly pressing public concern, resulting in efforts to reduce plastic waste, such as banning single-use plastic items, including drinking straws. Other efforts, such as increasing plastic recycling programs are limited by cost considerations and because most plastics can be recycled only a limited number of times before their physical properties become unsuitable for further use. Another option for addressing the issue of environmental buildup of plastic waste is to produce plastics that break down more quickly in the environment.

Biodegradable plastics are plastics that can be degraded by microorganisms into simple molecules, such as water, carbon dioxide, or methane and biomass in a much shorter time than required for typical plastics. Many biodegradable plastics can also be produced from renewable sources, rather than non-renewable petrochemical sources. However, biodegradable plastics are known to suffer from a number of undesirable characteristics, such as being brittle or having low thermal stability. Other known biodegradable plastics have a prohibitively high cost of production, which has deterred their widespread adoption.

Accordingly, there is a need for novel biodegradable plastics having improved mechanical characteristics. Additionally, there is a need for novel methods for producing biodegradable plastics from renewable raw materials to lower the cost of production.

Cannabis waste, and its disposal, is projected to become a significant challenge for the industry, as various jurisdictions begin to legalize the recreational use of cannabis. The production of one kilogram of cannabis for consumers results in eight kilograms of waste material. Current disposal methods for cannabis waste consist of strictly-regulated practices, including mixing the cannabis waste with chemicals and other materials for disposal.

Accordingly, there is a need to develop useful applications for the growing amounts of cannabis waste produced by this new industry.

### SUMMARY OF THE INVENTION

A biodegradable polymer composition, according to the present invention, comprises polyhydroxybutyrate and poly(3-hydroxybutyrate-co-3-hydroxyhexanoate) blended with thermoplastic starch, one or more compatibilizers selected from the group consisting of dihexyl sodium sulfosuccinate and maleic anhydride, and one or more additives selected from the group consisting of microcrystalline cellulose and cellulose.

In another embodiment, the biodegradable polymer composition comprises between 5 wt % and 70 wt % polyhydroxybutyrate, between 5 wt % and 70 wt % poly(3-hydroxybutyrate-co-3-hydroxyhexanoate), between 5 wt % and 45 wt % thermoplastic starch, between 0.5 wt % and 35 wt % of the one or more compatibilizers, and between 0.5 wt % and 15 wt % of the one or more additives.

In another embodiment, the biodegradable polymer composition comprises between 10 wt % and 30 wt % polyhydroxybutyrate, between 20 wt % and 60 wt % poly(3-hydroxybutyrate-co-3-hydroxyhexanoate), between 10 wt % and 30 wt % thermoplastic starch, between 10 wt % and 20 wt % of the one or more compatibilizers, and between 1 wt % and 10 wt % of the one or more additives.

In another embodiment, the biodegradable polymer composition comprises 20 wt % polyhydroxybutyrate, 40 wt % poly(3-hydroxybutyrate-co-3-hydroxyhexanoate), 20 wt % thermoplastic starch, 15 wt % of the one or more compatibilizers, and 5 wt % of the one or more additives.

According to another aspect of the present invention, a method of producing a biodegradable polymer, using cannabis waste as a carbon source, comprising the steps of: a) processing the cannabis waste by mechanical disruption; b) heating the cannabis waste in a mineral acid solution for at least 25 minutes at a temperature of at least 121° C., to produce a cannabis/acid solution; c) cooling, neutralizing, and filtering the cannabis/acid solution to produce a filtrate; d) mixing the filtrate with a mineral salt media in a ratio of between 1:1 and 1:2 to produce a production medium; e) inoculating the production medium with a starter culture of a microorganism selected from the group consisting of naturally occurring and engineered strains of Bacillus subtilis, Cupriavidus necator, Bacillus cereus, Bacillus brevis, Caulobacter cresentus, Bacillus sphaericus, Bacillus coagulans, Bacillus megaterium, Bacilllus circulans, Bacillus licheniformis, Escherichia coli, Microlanatus phosphovorous, Rhizobium meliloti, Rhizobium leguminosarumviciae, Bradyrhizobium japonicum, Burkholderia cepacia, Burkholderia sacchari, Cupriavidus necator, Neptunamonas Antarctica, Azobacter vinelandii, Pseudomonas putida, Pseudomonas aeruginosa, Aeromonas caviae, Aeromonas hydrophila, Aeromonas punctata, Alcaligenes latus, Halomonas boliviensis, Lactobacillus rhamnosus, and Fermicutes bacterium and incubating at a temperature of at least 30° C. for between 48 and 72 hours to produce a culture; and f) extracting a biodegradable polymer from the culture.

In another embodiment, the step of extracting a biodegradable polymer from the culture comprises the steps of: a) filtering the culture through a membrane with a pore size of about 1 mm; b) separating the cells of the microorganism from the filtered culture; c) suspending the cells in a NaOH solution and incubating at a temperature of at least 30° C. for at least 1.5 hours to release the biodegradable polymer from the cells; d) separating the biodegradable polymer from the NaOH solution and re-suspending the biodegradable polymer in water; e) separating the biodegradable polymer from the water and re-suspending the biodegradable polymer in an ethanol solution; and f) separating the biodegradable polymer from the ethanol solution.

According to another aspect of the present invention, a method of producing a production media from cannabis waste for use in producing a biodegradable polymer, comprises the steps of: a) processing raw cannabis waste by mechanical disruption to increase the available surface area of the cannabis waste; b) heating the cannabis waste in a mineral acid solution for at least 25 minutes at a temperature of at least 121° C., to produce a cannabis/acid solution; c) cooling, neutralizing, and filtering the cannabis/acid solution to produce a filtrate; and d) mixing the filtrate with a mineral salt media in a ratio of between 1:1 and 1:2.

In another embodiment, the method further comprises the steps of agitating the processed cannabis waste in water to break up the cannabis waste. Filtering the resulting mixture and then heating and stirring the filtrate in sodium hydroxide and hydrogen peroxide. Filtering the resulting slurry, neutralizing the pH and drying to produce a dried biomass, before the step of heating the cannabis waste in a mineral acid solution.

In another embodiment, the step of cooling, neutralizing, and filtering the cannabis/acid solution comprises stopping the reaction by adding cold deionised water. Centrifuging the resulting mixture and washing the precipitate with deionised water until a neutral pH is reached. Hydrolyzing the cellulose by acid hydrolysis at 0.5M at 70° C. in 67% zinc chloride and diluting the final product in sterile phosphate buffered saline.

According to another aspect of the present invention, a method of producing a biodegradable polymer comprises the steps of: a) inoculating nitrogen-limited production media having processed plant waste material as a carbon source with a starter culture of a microorganism selected from the group consisting of naturally occurring and engineered strains of Bacillus subtilis, Cupriavidus necator, Bacillus cereus, Bacillus brevis, Caulobacter cresentus, Bacillus sphaericus, Bacillus coagulans, Bacillus megaterium, Bacilllus circulans, Bacillus licheniformis, Escherichia coli, Microlanatus phosphovorous, Rhizobium meliloti, Rhizobium leguminosarum viciae, Bradyrhizobium japonicum, Burkholderia cepacia, Burkholderia sacchari, Cupriavidus necator, Neptunamonas Antarctica, Azobacter vinelandii, Pseudomonas putida, Pseudomonas aeruginosa, Aeromonas caviae, Aeromonas hydrophila, Aeromonas punctate, Alcaligenes latus, Halomonas boliviensis, Lactobacillus rhamnosus, and Fermicutes bacterium and incubating at a temperature of at least 30° C. for between 48 and 72 hours to produce a culture; b) filtering the culture through a membrane with a pore size of about 1 mm; c) separating the cells of the microorganism from the filtered culture; d) suspending the cells in a NaOH solution and incubating at a temperature of at least 30° C. for at least 1.5 hours to release the biodegradable polymer from the cells; e) separating the biodegradable polymer from the NaOH solution and re-suspending the biodegradable polymer in water; f) separating the biodegradable polymer from the water and re-suspending the biodegradable polymer in an ethanol solution; and g) separating the biodegradable polymer from the ethanol solution.

In another embodiment, the method produces PHB using a production media produced from cannabis waste and comprises the steps of growing one or more microorganisms capable of producing PHB in nutrient broth from stock. Inoculating the production media with the one or more microorganisms. Supplementing the production media with a limited nitrogen source and allowing the one or more microorganisms to grow in the production media. Centrifuging the production media to separate the cells of the one or more microorganisms from the production media and drying the cells. Re-suspending the dried cells in distilled water and adding sodium hydroxide to extract the PHB from the cells. Stopping the reaction by adjusting the pH to 7.0 and centrifuging the resulting mixture to separate out the PHB granules from the suspension. Rinsing the granules with distilled water and re-centrifuging the resulting mixture, as necessary. Separating the granules by the addition of a mineral acid and centrifuging the mixture. Discarding the liquid phase and washing the product in an alkaline bath to purify the PHB. Rinsing the PHB with water and centrifuging, as necessary.

### DESCRIPTION OF THE INVENTION

The present invention is directed to biodegradable polymer compositions and methods of producing the same. The biodegradable polymer compositions comprise polyhydroxybutyrate (PHB) and poly(3-hydroxybutyrate-co-3-hydroxyhexanoate) (PHBHHx) blended with thermoplastic starch (TPS), one or more compatibilizers, and one or more additives.

One or both of the PHB and PHBHHx used in the biodegradable polymer compositions described herein are preferably produced by microorganisms that are either naturally occurring or engineered to produce PHB and/or PHBHHx. The PHBHHx may be a random or non-random copolymer of PHB and HEN monomers. Preferably, the 3-hydroxyhexanoate units of the biosynthesized PHBHHx copolymer remain in the amorphous phase of the semi crystalline PHBHHx.

Suitable microorganisms for producing biodegradable polymers, including PHB and/or PHBHHx, include naturally occurring or engineered strains of: Bacillus subtilis, Cupriavidus necator, Bacillus cereus, Bacillus brevis, Caulobacter cresentus, Bacillus sphaericus, Bacillus coagulans, Bacillus megaterium, Bacilllus circulans, Bacillus licheniformis, Escherichia coli, Microlanatus phosphovorous, Rhizobium meliloti, Rhizobium leguminosarum viciae, Bradyrhizobium japonicum, Burkholderia cepacia, Burkholderia sacchari, Cupriavidus necator, Neptunamonas Antarctica, Azobacter vinelandii, Pseudomonas putida, Pseudomonas aeruginosa, Aeromonas caviae, Aeromonas hydrophila, Aeromonas punctate, Alcaligenes latus, Halomonas boliviensis, Lactobacillus rhamnosus, and Fermicutes bacterium. Preferably, an engineered strain of Bacillus subtilis, Cupriavidus necator, Lactobacillus rhamnosus, or Firmicutes bacterium is used to produce PHB and PHBHHx for the biodegradable polymer compositions, as described herein. Bacillus subtilis is preferred because it is a Gram positive bacteria and, therefore, does not contain toxic lipid A, which is present in Gram negative bacteria. Contamination of the biodegradable polymer with lipid A is undesirable in certain applications, such as in food packaging, medical devices or packaging, hygienic packaging, and products for small children.

Engineered microorganisms used in the methods described herein are genetically modified to express genes, including transgenes, necessary for production of one or more biodegradable polymers. Preferably, the biodegradable polymer produced is PHB. Suitable genes include one or more of the phaA, phaB, phaC, phaJ, and phaP genes encoding an acetyl-CoA acetyltransferase, an acetyl-CoA reductase, and PHB polymerase. Many microorganisms naturally express one or more of these genes. Some microorganisms may also express genes encoding one or more depolymerases that degrade one or more biodegradable polymers, including PHB. Preferably, an engineered microorganism used in the methods described herein would express the genes necessary for production of PHB and would not express any genes that encode a depolymerase capable of degrading PHB or any other desired biodegradable polymers produced by the selected microorganism.

Once synthesized and extracted, for example, according to one of the methods described herein, the PHB is blended with PHBHHx, thermoplastic starch, one or more compatibilizers, and one or more additives. The thermoplastic starch used in the biodegradable plastic compositions of the present invention is a plasticized natural polymer, preferably with low concentrations of ascorbic acid and citric acid, 30% glycerol as a plasticizer, and water at about 20 wt % with respect to the starch. Thermoplastic starch may be present in amounts of up to 45 wt % of the biodegradable polymer composition.

The thermoplastic starch may be prepared by any suitable method of preparing plasticized natural polymers, such as by mixing native starch with a plasticizer in a twin screw extruder at elevated temperatures of between about 30° C. to about 200° C. A mixture of water and glycerol is preferably used as the plasticizer. The plasticization of the thermoplastic starch can either be achieved prior to mixing of thermoplastic starch into the biodegradable polymer composition or by adding all the components at once (i.e. starch, glycerol, water, with the other components of the biodegradable polymer composition) to produce a final blend.

The compatibilizers may include one or more of dihexyl succinate, dihexyl sodium sulfosuccinate, maleic anhydride, methylene diphenyldiisocyanate, dioctyl fumarate, or other polar monomer grafted polyolefins. Preferably, both dihexyl succinate and maleic anhydride are present in the amount of 0.5-35 wt %.

The additives may include one or more of microcrystalline cellulose or cellulose. Preferably, both microcrystalline cellulose and cellulose are present in the amounts of 0.5-35 wt %.

The amount of time required for the biodegradable polymer compositions to break down may be selectively increased or decreased by manipulating the amounts of thermoplastic starch, microcrystalline cellulose, and/or cellulose in the compositions. As the relative amount of thermoplastic starch, microcrystalline cellulose, and/or cellulose increases, the time required for the compositions to break down decreases. Preferably, the relative amount of thermoplastic starch is adjusted in order to selectively increase or decrease the break down time of the compositions, rather than the relative amount of microcrystalline cellulose or cellulose. Further, the amount of time required for the biodegradable polymer compositions to break down may be selectively increased or decreased by manipulating the amounts of PHBHHx in the compositions. As the relative amount of PHBHHx increases, the time required for the compositions to break down increases.

The carbon source used for producing the biodegradable polymer may include: cannabis waste, leaves, fish solid waste, maple sap, pumpkin seeds, grape pomace or grape marc, or wine production/brewery/distillery waste. Preferably, cannabis waste material is used as a carbon source for the production of PHB. Cannabis waste consists of the roots, trimmings, leaves and stems of the plants, essentially every part except for the flowering bud of the Cannabis sativa L. plant.

Cannabis waste is particularly suitable for use as a carbon source in the production of PHB by microorganisms because the cannabis plant has a high biomass content and grows quickly in most climates with only moderate water and fertilizer requirements. Compared to other potential carbon sources, such as agricultural and forest biomass, coal, petroleum residues, and bones, cannabis waste has unique hierarchical pore structures and connected macropores. As a result, cannabis waste has desirable characteristics for use as a carbon source, including its porosity, adsorption capacity, and degree of surface reactivity. Relative to other potential carbon sources, cannabis waste also has a greater carbon concentration and lower nitrogen, potassium, and phosphorous content, which is favourable for production of PHB by microorganisms.

The cannabis waste is initially processed for use in the production of PHB by mechanical disruption, according to the following method. The raw cannabis waste may be processed by shredding, grinding, pressing, or other suitable means of mechanical disruption to increase the available surface area for the removal of cellulose and fatty acids. The separation of fatty acids from the processed cannabis waste is then performed to provide a carbon source for the synthesis of PHB, for example, as follows.

### Example: Production Media 1

The processed cannabis waste is mixed into a 1% sulphuric acid solution at a proportion of 10 g of plant waste per 100 mL of acidic solution. The solution is heated, preferably in an autoclave, for 25 minutes at 121° C., then cooled to room temperature. The solution is then neutralized with 2M NaOH solution and filtered through a sieve to remove larger particles of plant waste. The solution is then centrifuged for 20 minutes at 1500 g and the supernatant is filtered through a membrane having a pore size of about 1 mm. The resulting filtrate, a cannabis waste hydrolysate, may be immediately used or stored at 4° C. until needed.

The Production Media 1 is prepared by mixing the filtrate with 2× mineral salt media (0.9 g (NH₄)2SO₄, 0.3 g KH₂PO₄, 1.32 g Na₂HPO₄, 0.06 g MgSO₄.7H₂O, 300 uL of microelement solution (0.97 g FeCl₃, 0.78 g CaCl₂, 0.0156 g CuSO₄.5H₂O, 0.326 g NiCl₂.6H₂O in 100 mL of 0.1 M HCl)) in a ratio of 1:1. The media is autoclaved immediately for 10 minutes at 121° C.

### Example: Extraction 1

The synthesis and extraction of biodegradable polymer may be performed according to the following method. A suitable microorganism is grown in nutrient broth from a stock at 30° C. shaking at 150 rpm for 72 hours to produce a starter culture. After 72 hours, the starter culture is inoculated into Production Media 1 at 1/10 (v/v) and incubated at 30° C. shaking at 150 rpm for 72 hours to produce a culture.

The culture is then filtered through a membrane having a pore size of about 1 mm to remove insoluble plant matter. The cells of the microorganisms are then separated from the filtered culture by centrifugation at 1500 g for 20 minutes. The supernatant is discarded and the cells are then washed by resuspending the cells in mineral salt media and repeating the centrifugation and again discarding the supernatant.

The cells are then re-suspended in 150 mL of 0.2 M NaOH solution, vortexed vigorously to homogenize the solution and incubated at 30° C. for 1.5 hours. This causes the cells to lyse and releases the biodegradable plastic into the NaOH solution. The biodegradable polymer is then separated from the NaOH solution by centrifugation at 1500 g for 20 minutes and discarding the supernatant.

The biodegradable polymer is re-suspended in 150 mL of milliQ water and then separated from the water by centrifugation at 1500 g for 20 minutes. The supernatant is discarded to remove impurities. The biodegradable polymer is then re-suspended in 150 mL of 1% ethanol solution and separated from the ethanol solution by centrifugation at 1500 g for 20 minutes. The supernatant is again discarded to remove further impurities.

### Example: Production Media 2

Sonicate 5 g of plant waste with 300 mL deionised water at room temperature. Filter with Whatman No. 1 filter paper, then heat and vigorously stir the filtrate at 55° C. using 100 mL solution of sodium hydroxide (5%, w/v) and hydrogen peroxide (11%, v/v) for 90 min. Filter the slurry, neutralize the pH and dry at 50° C. Add 5 g of the dried biomass to 100 mL of 6 M sulphuric acid under vigorous stirring for 30 min and stop the reaction by adding 500 mL of cold deionised water. Centrifuge at 10,000 rpm for 10 min and wash with deionised water until neutral pH is achieved. Acquisition of simple monomers from cellulose is performed through the application of a 67% zinc chloride and acid hydrolysis at 0.5M and 70° C., this ideally results in a >80% yield of soluble sugars. The final glucose product is then diluted in 1 L sterile phosphate buffered saline pH 7.0, thereby producing Production Media 2 (Final concentrations: 8 g/L NaCl, 0.2 g/L KCl, 1.44 g/L Na₂HPO₄, 0.24 g/L K₂HPO₄).

### Example: Extraction 2

The synthesis and extraction of PHB for use in the biodegradable polymer compositions of the present invention may be performed according to the following method. A suitable Bacillus spp. is grown in nutrient broth from a stock overnight at 37° C. shaking at 120 rpm. A density of 1.5×10⁸ cells/mL can be added at 1/10 v/v to the Production Media 2 supplemented with a limited nitrogen source, such as Corn Steep Liquor (CSL) or an ammonium salt, at a concentration equivalent to 0.05% NH₄Cl and grown at 37° C. with 120 rpm shaking for 72 hours. The cells are then centrifuged at 6500 g for 10 min and dried at 50° C.

The dry cell mass can be measured and then PHB may be extracted using sodium hydroxide extraction and selective dissolution. Sodium hydroxide extraction is performed by re-suspending cells in distilled water and adding NaOH (0.2 N NaOH at 30C for 1-5 hours). Adjust pH to 7.0 with HCl to stop reaction. Centrifuge at 2500 g for 20 minutes. Recover the PHB granules by gently rinsing with distilled water, centrifuge again and air dry.

Selective dissolution is accomplished by applying a mineral acid, such as sulfuric acid, to the mixture resulting in granules separating in a solid phase while unwanted material is separated in a liquid phase. These phases can further be separated by centrifugation at 5000 g. The unwanted supernatant (liquid phase) is disposed of while the solid phase proceeds in processing. The mineral acid successfully isolates PHB from the mixture, but the purity is preferably improved before it is used. This is accomplished by washing the product in an alkaline bath, such as NaOH (pH 10). Following washing there would be a high yield and purity (>97%). To decolourize the product a commercially available bleach may be used. After a final centrifugation and a water rinse and the PHB product is ready for use.

To measure the production of PHB, centrifuge and wash the pellet with alcohol. Dissolve the pellet in chloroform and transfer to clean and pre-weighed serum tubes. Allow the chloroform to evaporate and weigh the tubes to calculate the amount of PHB obtained. The present method may produce between 2-5 g/L of PHB in yield from a 7-9 g/L dry cell mass. This method of growth can be adapted to use with Bacillus spp. to also generate larger amounts of PHB from less volume of culture media and less time. Alternatively, a similarly engineered strain of Cupriavidus necator may be used instead.

### Example: Extraction 3

In another embodiment, PHB may be synthesized and extracted, using Cupriavidus necator as the microorganism and cannabis waste as the carbon source, according to the following method. A strain of C. necator that is able to produce PHB is used, referred to as Alcaligenes eutrophus H16 (C. necator was formerly known as Alcaligenes eutrophus). The A. eutrophus H16 is cultured at 30° C. in a nitrogen limited mineral salt medium with 1% (v/v) cannabis oil and 0.05% (w/v) NH₄Cl for 72 hours. Kanamycin (50 mg/L) is added to maintain the broad-host range plasmid inserted in A. eutrophus H16. After growth, the cells are harvested and washed twice with distilled water and lyophilized. The PHB is extracted using hot chloroform in a Soxhlet apparatus and purified by methanol reprecipitation.

PHB may be produced by the method of Extraction 3, at a rate of about 0.0128 g PHB per g hemp oil per hour.

### Example: Extraction 4

In another embodiment, PHB may be synthesized and extracted, using C. necator as the microorganism and cannabis waste as the carbon source, according to the following method. Optionally, the surfactant gum arabic may be added to the reaction media to enhance C. necator's ability to interact/utilize the cannabis oil, as it is non-toxic and does not inhibit the growth of C. necator. C. necator may be grown from stock in a minimal medium containing 2% fructose and 0.1% NH₄Cl (16 g/L), NaH₂PO₄ (4 g/L), Na₂HPO₄ (4.6 g/L), K₂SO₄ (0.45 g/L), MgSO₄ (0.39 g/L), CaCl₂ (62 mg/L), and 1 ml/L of a trace element solution (15 g/L FeSO₄.7H₂O, 2.4 g/L MnSO₄.H₂O, 2.4 g/L ZnSO₄.7H₂O, and 0.48 g/L CuSO₄.5H₂O dissolved in 0.1 M hydrochloric acid). Cells from the minimal media are used to inoculate each fermenter to reach an OD600 of 0.1. Each reaction vessel contains 400 mL of emulsified cannabis oil medium. For minimal medium with 0.1% NH₄Cl use approximately 2% cannabis oil. To prepare the medium, use a 10× solution of gum arabic mixed in water and stirred rapidly. Centrifuge at 10,500 g to separate out insoluble particles. Water, clarified gum arabic solution, and cannabis oil are combined with the sodium phosphate (4.0 g/L) and K₂SO₄ (0.45 g/L). Emulsify the mixture through homogenization or sonication. The amount of water added before emulsification depends on the particular apparatus used to make the emulsion. After emulsification, autoclave, cool and add MgSO₄ (0.39 g/L), CaCl₂ (62 mg/L), trace elements (15 g/liter FeSO₄.7H₂O, 2.4 g/liter MnSO₄.H₂O, 2.4 g/liter ZnSO₄.7H₂O, and 0.48 g/liter CuSO₄.5H₂O dissolved in 0.1 M hydrochloric acid), and gentamicin (10 μg/mL). Each reaction vessel is maintained at 30° C., with a pH of 6.8 (controlled with 2 M NaOH) and stirred at a rate of 500-900 rpm with a dissolved oxygen concentration of 40% for 72 hours. Preferably, a fed batch culture technique is used to maintain an excess of carbon in order to increase PHB production.

PHB may be produced by the method of Extraction 4, at a rate of about 0.2415 g PHB per g cannabis oil.

### Example: Extraction 5

In another embodiment, PHB may be synthesized and extracted, using a mixture of Cupriavidus necator and an engineered strain of Escherichia coli, and, optionally, an engineered strain of Aeromonas hydrophila having the phbA and phbB genes, as the microorganisms and cannabis waste as the carbon source, according to the following method. First, the cannabis waste is shredded and placed into water at about 2% (w/v) and at a temperature of about 30° C. The cannabis-water mixture is inoculated with the mixed culture of C. necator and E. coli and, optionally, A. hydrophila and fertilizer is added, such as rice bran extract at 0.1%. The reaction medium is then stirred for 20 hours to permit growth. After the initial growth period, the reaction medium is stirred for a further 15 hours, without the addition of any further fertilizer to induce a state of nitrogen deprivation and promote production of PHB.

The extraction of PHB is accomplished by adding a mineral acid, such as sulfuric acid, to the reaction medium after about 35 hours. The PHB granules are separated by centrifugation at 5000 g. The unwanted supernatant (liquid phase) is disposed of while the solid phase proceeds in processing. The mineral acid isolates PHB from the mixture. The purity of the compounds may be improved by washing in an alkaline bath, such as NaOH (pH 10), followed by a final centrifugation and a water rinse. The method provides a high yield and purity of PHB (>97%). Optionally, a commercially available bleach may be used to decolourize the product.

### Example: Production Media 3

In another embodiment, PHB may be synthesized and extracted, using Pseudomonas putida GPp104 as the microorganisms and cannabis waste as the carbon source, according to the following method. The P. putida is grown overnight in LB media containing 50 mg/L kanamycin at 30° C. shaking at 200 rpm. The phosphate buffered saline solution for cultivating this strain is composed of 9.0 g/L Na₂HPO₄.12H₂O, 1.5 g/L KH₂PO₄, 1.0 g/L (NH₄)₂SO₄, and 0.4 g/L MgSO₄.7-H₂O with a pH of 7.0.

### Example: Extraction 6

A density of 1.5×10⁸ cells/mL of the overnight P. putida culture can be added at 1/10 v/v to 1 L of Production Media 3 and grown at 30° C. with 200 rpm shaking for 72 hours. The PHB can be extracted using sodium hypochlorite as follows. To 8 g biomass, add 100 mL sodium hypochlorite (30%) and incubate for 90 min at 37° C. Centrifuge and wash the pellet with alcohol. Dissolve the pellet in chloroform and, optionally, transfer to clean and pre-weighed serum tubes. Allow the chloroform to evaporate and weigh the tubes to calculate the amount of PHB obtained.

The present invention has been described with reference to an exemplary embodiment, however, it will be understood by those skilled in the art that various changes may be made, and equivalents may be substituted for elements thereof, without departing from the scope of the invention as set out in the following claims. Therefore, it is intended that the invention not be limited to the particular embodiments disclosed herein.

What is claimed is:

1. A method of producing a biodegradable polymer, using cannabis waste as a carbon source, comprising the steps of:
a. processing the cannabis waste by mechanical disruption;
b. heating the cannabis waste in a mineral acid solution for at least 25 minutes at a temperature of at least 121° C., to produce a cannabis/acid solution;
c. cooling, neutralizing, and filtering the cannabis/acid solution to produce a filtrate;
d. mixing the filtrate with a mineral salt media in a ratio of between 1:1 and 1:2 to produce a production medium;
e. inoculating the production medium with a starter culture of a microorganism selected from the group consisting of naturally occurring and engineered strains of Bacillus subtilis, Cupriavidus necator, Bacillus cereus, Bacillus brevis, Caulobacter cresentus, Bacillus sphaericus, Bacillus coagulans, Bacillus megaterium, Bacilllus circulans, Bacillus licheniformis, Escherichia coli, Microlanatus phosphovorous, Rhizobium meliloti, Rhizobium lefuminosarum viciae, Bradyrhizobium japonicum, Burkholderia cepacia, Burkholderia sacchari, Cupriavidus necactor, Neptunamonas Antarctica, Azobacter vinelandii, Pseudomonas putida, Pseudomonas aeruginosa, Aeromonas caviae, Aeromonas hydrophila, Aeromonas punctata, Alcaligenes latus, Halomonas boliviensis, Lactobacillus rhamnosus, and Fermicutes bacterium and incubating at a temperature of at least 30° C. for between 48 and 72 hours to produce a culture; and
f. extracting a biodegradable polymer from the culture.

2. The method of claim 1, wherein the step of extracting a biodegradable polymer from the culture comprises the steps of:
a. filtering the culture through a membrane with a pore size of about 1 mm;
b. separating the cells of the microorganism from the filtered culture;
c. suspending the cells in a NaOH solution and incubating at a temperature of at least 30° C. for at least 1.5 hours to release the biodegradable polymer from the cells;
d. separating the biodegradable polymer from the NaOH solution and re-suspending the biodegradable polymer in water;
e. separating the biodegradable polymer from the water and re-suspending the biodegradable polymer in an ethanol solution; and
f. separating the biodegradable polymer from the ethanol solution.

3. The method of claim 2, wherein the biodegradable polymer is polyhydroxybutyrate.

4. The method of claim 3, wherein the microorganism does not express a gene encoding a depolymerase capable of degrading polyhydroxybutyrate.

5. The method of claim 4, wherein the microorganism is an engineered strain of Bacillus subtilis that expresses one or more genes encoding an acetyl-CoA acetyltransferase, an acetyl-CoA reductase, and polyhydroxybutyrate polymerase.

6. The method of claim 5, wherein the one or more genes are selected from the group consisting of phaA, phaB, phaC, phaJ, phaP.

7. The method of claim 4, wherein the microorganism is an engineered strain of Cupriavidus Necator that expresses one or more genes encoding an acetyl-CoA acetyltransferase, an acetyl-CoA reductase, and polyhydroxybutyrate polymerase.

8. The method of claim 7, wherein the one or more genes are selected from the group consisting of phaA, phaB, phaC, phaJ, phaP.

9. A method of producing a production media from cannabis waste for use in producing a biodegradable polymer, comprising the steps of:
a. processing the cannabis waste by mechanical disruption;
b. heating the cannabis waste in a mineral acid solution for at least 25 minutes at a temperature of at least 121° C., to produce a cannabis/acid solution;
c. cooling, neutralizing, and filtering the cannabis/acid solution to produce a filtrate; and
d. mixing the filtrate with a mineral salt media in a ratio of between 1:1 and 1:2.

10. A method of producing a biodegradable polymer, comprising the steps of:
a. inoculating nitrogen-limited production media having processed plant waste material as a carbon source with a starter culture of a microorganism selected from the group consisting of naturally occurring and engineered strains of Bacillus subtilis, Cupriavidus necator, Bacillus cereus, Bacillus brevis, Caulobacter cresentus, Bacillus sphaericus, Bacillus coagulans, Bacillus megaterium, Bacilllus circulans, Bacillus licheniformis, Escherichia coli, Microlanatus phosphovorous, Rhizobium meliloti, Rhizobium leguminosarum viciae, Bradyrhizobium japonicum, Burkholderia cepacia, Burkholderia sacchari, Cupriavidus necactor, Neptunamonas Antarctica, Azobacter vinelandii, Pseudomonas putida, Pseudomonas aeruginosa, Aeromonas caviae, Aeromonas hydrophila, Aeromonas punctata, Alcaligenes latus, Halomonas boliviensis, Lactobacillus rhamnosus, and Fermicutes bacterium and incubating at a temperature of at least 30° C. for between 48 and 72 hours to produce a culture;
b. filtering the culture through a membrane with a pore size of about 1 mm;
c. separating the cells of the microorganism from the filtered culture;
d. suspending the cells in a NaOH solution and incubating at a temperature of at least 30° C. for at least 1.5 hours to release the biodegradable polymer from the cells;
e. separating the biodegradable polymer from the NaOH solution and re-suspending the biodegradable polymer in water;
f. separating the biodegradable polymer from the water and re-suspending the biodegradable polymer in an ethanol solution; and
g. separating the biodegradable polymer from the ethanol solution.

11. The method of claim 10, wherein the biodegradable polymer is polyhydroxybutyrate.

12. The method of claim 11, wherein the microorganism does not express a gene encoding a depolymerase capable of degrading polyhydroxybutyrate.

13. The method of claim 12, wherein the microorganism is an engineered strain of Bacillus subtilis that expresses one or more genes encoding an acetyl-CoA acetyltransferase, an acetyl-CoA reductase, and polyhydroxybutyrate polymerase.

14. The method of claim 13, wherein the one or more genes are selected from the group consisting of phaA, phaB, phaC, phaJ, phaP.

15. The method of claim 12, wherein the microorganism is an engineered strain of Cupriavidus Necator that expresses one or more genes encoding an acetyl-CoA acetyltransferase, an acetyl-CoA reductase, and polyhydroxybutyrate polymerase.

16. The method of claim 15, wherein the one or more genes are selected from the group consisting of phaA, phaB, phaC, phaJ, phaP.

17. A biodegradable polymer, comprising between 5 wt % and 70 wt % polyhydroxybutyrate, between 5 wt % and 70 wt % poly(3-hydroxybutyrate-co-3-hydroxyhexanoate), between 5 wt % and 45 wt % thermoplastic starch, between 0.5 wt % and 35 wt % of one or more compatibilizers, and between 0.5 wt % and 15 wt % of one or more additives.

18. The biodegradable polymer of claim 17, wherein the one or more compatibilizers are selected from the group consisting of dihexyl succinate, dihexyl sodium sulfosuccinate, maleic anhydride, methylene diphenyldiisocyanate, and dioctyl fumarate and the one or more additives are selected from the group consisting of microcrystalline cellulose and cellulose.

19. The biodegradable polymer of claim 17, wherein the one or more compatibilizers are selected from the group consisting of dihexyl sodium sulfosuccinate and maleic anhydride and the one or more additives are selected from the group consisting of microcrystalline cellulose and cellulose.

20. The biodegradable polymer of claim 17, wherein the one or more compatibilizers are dihexyl sodium sulfosuccinate and maleic anhydride and the one or more additives are microcrystalline cellulose and cellulose.

21. The biodegradable polymer of claim 20, wherein the thermoplastic starch is a plasticized natural polymer comprising about 30 wt % glycerol as a plasticizer and about 20 wt % water.

22. The biodegradable polymer of claim 21, comprising between 20 wt % and 60 wt % poly(3-hydroxybutyrate-co-3-hydroxyhexanoate), between 10 wt % and 30 wt % thermoplastic starch, between 10 wt % and 20 wt % of the one or more compatibilizers, and between 1 wt % and 10 wt % of the one or more additives.

23. The biodegradable polymer of claim 21, comprising 20 wt % polyhydroxybutyrate, 40 wt % poly(3-hydroxybutyrate-co-3-hydroxyhexanoate), 20 wt % thermoplastic starch, 15 wt % of the one or more compatibilizers, and 5 wt % of the one or more additives.

24. The use of cannabis waste as a carbon source for producing a biodegradable polymer.

25. The use of claim 24, wherein the cannabis waste consists of one or more of the roots, trimmings, leaves, stalks, and stems of the cannabis plant.

26. The use of claim 25, wherein the biodegradable polymer is polyhydroxybutyrate.
